(19) United States
(12) Patent Application Publication
(10) Pub. No.: US 2004/0162503 A1
(43) Pub. Date: Aug. 19, 2004

(54) Method for tissue-selective treatment in therapy and surgery

(76) Inventors: Steffen Dubnack, Jena (DE); Dirk Preuss, Jena (DE)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/773,669

(22) Filed: Feb. 6, 2004

(30) Foreign Application Priority Data
Feb. 7, 2003 (DE) 103 05 062.0

(51) Int. Cl. A61B 5/05

(52) U.S. Cl. 600/554

(57) ABSTRACT

The present invention is directed to a surgical method, particularly for procedures on the brain, in which it is possible to select the tissue to be treated. In the method according to the invention, a probe is positioned in the area of the diseased change and tissue selection is activated in that different electrical and/or electro-magnetic stimulus signals which can be preadjusted or modulated are sent to the tissue. Healthy tissue parts can be distinguished from pathologically changed tissue parts by evaluating the responses to these stimuli. When the stimulus response is absent or unexpected, the corresponding therapeutic or surgical treatment is carried out by means of the same probe at the selected site. Otherwise, the probe is repositioned and the tissue selection is activated again. With the method according to the invention for tissue-selective treatment, particularly on the brain, it is possible to sever, fragment and/or suction off certain pathologically altered tissue parts from the remaining, healthy tissue parts without damaging the healthy tissue parts.

### CROSS-REFERENCE TO RELATED APPLICATION

This application claims priority of German Application No. 103 05 062.0, filed Feb. 7, 2003, the complete disclosure of which is hereby incorporated by reference.

### BACKGROUND OF THE INVENTION

a) Field of the Invention

The present invention is directed to a surgical method, particularly for therapeutic and surgical procedures on the brain, which makes it possible to select the tissue to be treated.

b) Description of the Related Art

According to the known prior art, electrosurgical instruments are very common in medical technology. They are used for thermal coagulation, for cutting and for fragmenting tissue in different body parts and organs. Separating diseased tissue types (e.g., tumors) from healthy tissue types (e.g., nerves, tendons) is particularly important when applied to the brain. The diseased tissue must be fragmented and removed by suction without damaging the healthy tissue because damage to or destruction of the facial nerve or speech center, for example, could cause grave injury to the patient. Brain operations are made more difficult in that they must usually be carried out using endoscopes and it is not possible to carry out a biopsy in advance. Since it is unlikely that the observer will be able to distinguish visually between diseased tissue and healthy tissue, numerous solutions are known from the prior art in which the surgical instruments possess means for tissue differentiation.

DE 195 42 419, for example, describes a high-frequency generator for high-frequency surgery with tissue differentiation. This technique is applied particularly for removal of tissue parts when the operation site can only be reached through natural or small artificial body openings. Correct metering of instantaneously applied power presents a great problem in high-frequency surgery. The high-frequency output required for cutting may vary to a great degree because it is dependent upon factors such as tissue quality and water content, cutting speed and cutting depth, etc. Inevitably, this also results in differences in the quality of the cut surfaces which must be eliminated through a measuring and regulating device for adapting the laser output to the tissue to be cut. However, tissue differentiation remains difficult in spite of a large variety of electrical signals that can be detected by measurement technique. Tissue differentiation cannot begin until as many tissue-specific characteristic values as possible are combined.

The arrangement for cutting biological tissue according to U.S. Pat. No. 5,749,869 is likewise based on high-frequency current and permits tissue differentiation in the vicinity of the operating site. In this way, it is possible to adapt the generator to the goal of the operation. In order to differentiate between different materials, the different electrophysical characteristics of these materials which influence the arc required for cutting are observed. The spectral output distribution of the generator signal which is influenced in this way is evaluated by means of two filters and serves as a basis for adjusting new reference values of the generator.

However, the solutions mentioned above are disadvantageous in that the differentiation of tissue is first carried out directly during the cutting or coagulation process. Accordingly, cautious, slow cutting is absolutely necessary in areas of tissue transitions in order to be able to interrupt the cutting or coagulation process in a timely manner.

The solution described in Patent WO 93/03679 is also directed to a high-frequency surgical generator for regulated coagulating cutting. The solution comprises a regulating device for adjusting the electrical output values and a device for determining the state of the tissue in the vicinity of the cutting electrode. The purpose of determining the tissue state is to determine the required mode of operation, i.e., cutting or coagulation, for further treatment. For this purpose, the treatment process is divided into corresponding time intervals. However, this solution is unsuitable for tissue differentiation, for example, differentiating between diseased and healthy or normal tissue.

The invention according to EP 0 599 007 is directed to an arrangement and a method for selective cutting of biological tissue with a pulsating liquid jet. For this purpose, light pulses from a pulsed laser are conducted into a tube filled with liquid; the light pulses which are absorbed therein cause the acceleration of a liquid column in the direction of the tissue to be cut. Adjustment of the cutting power prevents injury to blood vessels when severing tissue parts, these blood vessels generally being more robust. Tissue differentiation in the true sense is also impossible with this solution. Exact regulation of the cutting power is also extremely difficult.

While the technical solutions cited above are suitable for cutting, coagulating and/or fragmenting at operating sites which can only be reached through natural or small artificial body openings, they are not suitable for differentiating, e.g., between the diseased and healthy tissue.

### OBJECT AND SUMMARY OF THE INVENTION

The primary object of the present invention is to develop an improved therapeutic and surgical method, particularly for procedures on the brain, in which it is possible to differentiate between tissue.

According to the invention, this object is met by a method for tissue-selective treatment in therapy and surgery comprising the steps of positioning a probe in the area of the diseased change after placing on the body organ or body tissue to be treated, activating tissue selection in that different electrical and/or electromagnetic stimulus signals which can be preadjusted or modulated are sent to the tissue in order to stimulate the latter, distinguishing the healthy tissue parts from the pathologically changed tissue parts by evaluating the responses to these stimuli; wherein, in the case of an expected stimulus response identifying healthy tissue, repositioning the probe and activating the tissue selection again, or when the stimulus response identifying pathologically altered tissue is absent or unexpected, carrying out the corresponding therapeutic or surgical treatment by the same probe at the selected site.

The method is based on the direct selection of tissue before, during and/or after treatment and combines a therapeutic and diagnostic method in only one probe. By leaving the probe at the application site, time-consuming exchange and re-positioning of the probe can be dispensed with. Substantial disadvantages of the known prior art are overcome with the method.

The method according to the invention for tissue-selective therapeutic and surgical treatment will be described in the following with reference to an embodiment example.

The method provides for the use of a probe with at least one electrically acting connection element in order to produce a direct or indirect (by way of an isotonic saline solution) connection to the tissue to be treated. The method is generally applicable when this condition is ensured. However, electrosurgical instruments such as “ArthroCare”, “FugoBLADE” or the pulsed, bipolar plasma scalpel (pulsed electron avalanche knife) offered by Carl Zeiss Jena GmbH can be advantageously used for cutting and fragmenting tissue.

But the method according to the invention can also be applied when using surgical instruments based on laser radiation or other energy sources. The total arrangement for carrying out the method must be correspondingly adapted to the different pulse shapes that are used, for example, electrical pulses for tissue selection and light pulses for therapeutic and surgical treatment.

In the method for tissue-selective treatment in therapy and surgery, a probe is positioned in the area of the diseased change after placing on the body organ or body tissue to be treated and tissue selection is activated. The positioning of the probe can be carried out visually by means of endoscopes or other methods such as MRT, CTG, etc. based on diagnostic examinations carried out beforehand.

Tissue selection is carried out in that different electrical and/or electromagnetic stimulus signals which can be preadjusted or modulated are sent to the tissue of the affected region. Healthy tissue parts are distinguished from diseased altered tissue parts by evaluating the stimulus responses. The following changes or influences can be used, for example, to evaluate the stimulus signals sent to the tissue: changes in the EKG or EGG; affect on sight, hearing or speech; muscular tremors; movements of extremities or body parts; affect on equilibrium; changes in cardiovascular system, e.g. changes in heart rate or fibrillation; and affect on memory, logical thought or motor skills.

In case of an expected stimulus response identifying healthy tissue, the probe is repositioned and the tissue selection is activated again. Tissue selection during the repositioning of the probe due to an expected stimulus response identifying healthy tissue can be carried out by iterative or continuous transmission of stimulus signals.

In contrast, when the stimulus response identifying pathologically altered tissue is absent or unexpected, the corresponding therapeutic or surgical treatment is carried out by means of the same probe at the selected site.

In another arrangement of the method according to the invention for tissue-selective treatment, a kind of ONLINE tissue selection can be carried out by means of alternating treatment and positioning with tissue selection and immediate evaluation of the stimulus responses. For this purpose, it must be insured that the tissue selection is realized in the direction of the cutting movement in front of the cutter so that healthy tissue is not severed during the cutting movement. Further, it is also possible to warn the user about critical tissue areas during treatment and/or to interrupt treatment. It is also possible to warn the user when the cutting movement is too fast or to interrupt the process.

In the method according to the invention, it is not important whether the instrument which transmits the stimuli also detects and further processes the stimulus responses itself or whether other systems are used for detecting and processing.

In the method, according to the invention, for tissue-selective treatment in therapy and surgery, particularly on the brain, it is possible to sever, fragment and/or suction certain pathologically altered tissue parts (e.g., tumors) from the remaining, healthy tissue parts (e.g. nerves, tendons) without injuring the healthy tissue parts. Damage to or destruction of certain types of tissue such as the facial nerve, speech center, etc. can result in grave injury to the patient. By leaving the probe at the site of application, time-consuming exchange of the probe and repositioning of the probe can be dispensed with. The method is based on a combination of therapeutic and diagnostic methods in one probe and tissue selection immediately before, during and after treatment.

While the foregoing description and drawings represent the present invention, it will be obvious to those skilled in the art that various changes may be made therein without departing from the true spirit and scope of the present invention.

What is claimed is:

1. A method for tissue-selective treatment in therapy and surgery comprising the steps of:
positioning a probe in the area of the diseased change after placing on the body organ or body tissue to be treated;
activating tissue selection in that different electrical and/or electromagnetic stimulus signals which can be preadjusted or modulated are sent to the tissue in order to stimulate the latter;
distinguishing the healthy tissue parts from the pathologically changed tissue parts by evaluating the responses to these stimuli;
wherein, in the case of an expected stimulus response identifying healthy tissue, repositioning the probe and activating the tissue selection again, or when the stimulus response identifying pathologically altered tissue is absent or unexpected, carrying out the corresponding therapeutic or surgical treatment by the same probe at the selected site.

2. The method for tissue-selective treatment according to claim 1, in which the tissue selection during the repositioning of the probe due to an expected stimulus response identifying healthy tissue can be carried out by iterative or continuous transmission of stimulus signals.

3. The method for tissue-selective treatment according to claim 1, wherein a direct online tissue selection is carried out by alternating treatment and positioning with tissue selection and immediate evaluation of the stimulus responses and the user is warned during treatment of critical tissue regions and/or the therapy can be interrupted.
